(19) United States
(12) Patent Application Publication
(10) Pub. No.: US 2024/0382686 A1
(43) Pub. Date: Nov. 21, 2024

(54) Electronic Module, Drug Delivery Device and Method for Operating an Electronic Module

(71) Applicant: Sanofi, PARIS (FR)

(72) Inventors: Hester Jane Corne, Warwick, Warwickshire (GB); John Whitehouse, Harvington Evesham (GB)

(21) Appl. No.: 18/692,220

(22) PCT Filed: Sep. 22, 2022
(86) PCT No.: PCT/EP2022/076292

§ 371 Date: Mar. 14, 2024

(30) Foreign Application Priority Data
Sep. 24, 2021 (EP) 21315181.4

(51) Int. Cl. A61M 5/315; G08C 17/02; G16H 20/17

(52) U.S. Cl. CPC A61M 5/31546; G16H 20/17; A61M 2205/3327; A61M 2205/3584; A61M 2205/52; A61M 2205/581; A61M 2205/582; A61M 2205/583; A61M 2205/8206; G08C 17/02

(57) ABSTRACT

An electronic module for a drug delivery device comprises a dose setting and drive mechanism configured to perform a dose setting operation for setting a dose to be delivered by the drug delivery device and a dose delivery operation. The module comprises a processor, a sensor arrangement, a communication unit with a wireless communication interface connected to the processor and operable to establish communication with another device and to transfer data to the other device, an electronic user feedback generator, a memory, and a power source. The processor is configured to prevent data transfer by the communication interface during operation of the electronic user feedback generator and/or of the sensor arrangement, and/or limit the amount of data transferred by the communication interface at any one time by limiting the packet size of the data transferred by the communication interface, and/or limit the rate at which data is transferred.

### CROSS-REFERENCE TO RELATED APPLICATIONS

The present application is the national stage entry of International Patent Application No. PCT/EP2022/076292, filed on Sep. 22, 2022, and claims priority to Application No. EP 21315181.4, filed on Sep. 24, 2021, the disclosures of which are incorporated herein by reference.

### TECHNICAL FIELD

The present disclosure is generally directed to an electronic system, e.g., a module, for a drug delivery device. The present disclosure further relates to a drug delivery device, which preferably comprises such an electronic module. Still further, the present disclosure is directed to a method for operating such an electronic module.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present disclosure is applicable for disposable and reusable devices.

For such devices the functionality of recording doses that are dialed and/or delivered from the pen may be of value to a wide variety of device users as a memory aid or to support detailed logging of dose history. Thus, drug delivery devices using electronics are becoming increasingly popular in the pharmaceutical industry as well as for users or patients. An example of a medical device communicating with a data management device via Bluetooth® is known from WO 2020/193090 A1.

For example, a drug delivery device is known from EP 2 814 545 A1 comprising an electronic clip-on module. The clip-on module comprises a battery which powers a processor and further components controlled by the processor, like light-sources, a photometer, an acoustic sensor, an acoustical signal generator and a wireless unit, like a Bluetooth® transceiver, configured to transmit and/or receive information to/from another device in a wireless fashion.

However, especially if the device is designed to be self-contained, that is to say without a connector for a connection to an electrical power source which is necessary to provide electrical power for the operation of the device, the management of the resources of a power supply integrated into the device is particularly important.

WO 2021/191322 A1 and WO 2021/191327 A1 disclose advantageous embodiments of electronic systems for drug delivery devices with improved power management. These electronic systems comprise a switch assembly for activating/deactivating power consuming functions of the electronic systems.

Such drug delivery devices are typically manufactured in large scale such that an efficient and simple assembly is an important issue to keep production costs reasonably low.

### SUMMARY

It is an object of the present disclosure to provide improvements for electronic modules to be used with or being integrated in drug delivery devices allowing increased life of the module when used e.g. with a non-rechargeable, non-user replaceable power source, like a coin-cell or similar battery.

This objective is solved for example by the subject matter defined in the independent claims. Advantageous embodiments and refinements are subject to the dependent claims. However, it should be noted that the disclosure is not restricted to the subject matter defined in the appended claims. Rather, the disclosure may comprise improvements in addition to or as an alternative to, the ones defined in the independent claims as will become apparent from the following description.

One aspect of the disclosure relates to an electronic module suitable for use with and/or in a drug delivery device. Such a drug delivery device may comprise a dose setting and drive mechanism which is configured to perform a dose setting operation for setting a dose to be delivered by the drug delivery device and a dose delivery operation for delivering the set dose.

The electronic module preferably comprises at least one processor, e.g., a microcontroller, a sensor arrangement, a communication unit with a wireless communication interface, at least one electronic user feedback generator, a memory for storing measurement data, and a power source connected to the at least one microcontroller. According to an aspect of the present disclosure, the at least one processor is configured to prevent data transfer by the communication interface during operation of the at least one electronic user feedback generator and/or during operation of the sensor arrangement. In addition or as an alternative, the at least one processor may be configured to limit the amount of data transferred by the communication interface at any one time by limiting the packet size of the data transferred by the communication interface. In addition or as an alternative, the at least one processor may be configured to limit the rate at which data is transferred by the communication interface. For example, according to an aspect of the present disclosure, the at least one processor is configured to prevent data transfer by the communication interface during operation of the at least one electronic user feedback generator and/or during operation of the sensor arrangement and the at least one processor is configured to limit the amount of data transferred by the communication interface at any one time by limiting the packet size of the data transferred by the communication interface and/or to limit the rate at which data is transferred by the communication interface.

If the electronic module is fitted with a small non-rechargeable, non-user replaceable, coin cell the overall power available over the life of the device is limited. It is typical of cells of this type that they cannot sustain large and prolonged current flows. If large current flows are present, then the supply voltage of the cell can be seen to drop. A similar behaviour can be observed for rechargeable power sources. The present disclosure is based on the idea of pacing activities of the electronic module involving large current flows to avoid undesired voltage drops.

The sensor arrangement may be connected to the at least one processor and operable to generate measurement data indicative of the dose setting operation and/or the dose delivery operation. The sensor arrangement may comprise one or more electrical switch(es) and/or may include optical and/or capacitive and/or acoustic sensors for detecting a movement of one or more component parts of the dose setting and drive mechanism of the drug delivery device. In one example, the sensor arrangement comprises at least one light source, e.g., an LED, and at least one light sensor, e.g. a photo detector. The sensor arrangement may be part of an encoding or motion sensing unit designed and working as described in unpublished EP 20315066.9 and EP 20315357.2, the disclosure of which is incorporated herein by reference.

The at least one electronic user feedback generator may be connected to the at least one processor and operable to generate a feedback signal. For example, the at least one electronic user feedback generator may comprise at least one light source, e.g., an LED, at least one sounder (i.e. an acoustical signal generator) and/or at least one vibration generator, e.g., a vibration motor.

The communication unit with the wireless communication interface may be connected to the at least one processor and operable to establish communication with another device and to transfer data to another device. Despite the fact that establishing wireless communication typically involves a transfer of data, with respect to the present disclosure, establishing communication, which may include for example the process of broadcasting advertising packets, scanning for such advertising packets and pairing two devices, is to be distinguished from the data transfer itself which is defined to occur only after successful pairing and typically involves significantly higher data transfer volume compared to establishing wireless communication like a advertising and/or a pairing.

According to an aspect of the present disclosure, the at least one processor is configured to prevent any data transfer by the communication interface during operation of the at least one electronic user feedback generator and/or during operation of the sensor arrangement, i.e., the data transfer for establishing communication (advertising and/or a pairing) as well as the transfer of measurement data and/or dose data. As an alternative, establishing communication (advertising and/or a pairing) could be permitted due to the significally lower data transfer volume and, thus an only moderate voltage drop caused, while preventing the transfer of measurement data and/or dose data.

In an example of the present disclosure, all data transfers, i.e., any communication activity (including e.g., pairing) are prevented during LED activity. Manual synchronisation and/or pairing events also transfer data (partially more than the transfer of dose records, actually) and, thus, are preferably paced as well. The method may even be used during Bluetooth® Low Energy (BLE) advertising. In an exemplary embodiment, BLE activity and activity of optical sensors never occur at the same time although from a power perspective, this would work quite well. As an LED used as electronic user feedback generator may draw significantly more power than sensor LEDs, the at least one processor is preferably configured to prevent data transfer by the communication interface during operation of the at least one electronic user feedback generator.

The communication unit for communicating with another device may comprise a wireless communications interface for communicating with another device via a wireless network such as Wi-Fi or Bluetooth®. In addition, the communication unit may comprise an interface for a wired communications link, such as a socket for receiving a Universal Serial Bus (USB) mini-USB or micro-USB connector. Preferably, the electronic system comprises an NFC, WiFi and/or Bluetooth® unit as the communication unit. The communication unit may be provided as a communication interface between the module or the drug delivery device and the exterior, such as other electronic devices, e.g., mobile phones, personal computers, laptops and so on. For example, measurement data, i.e., dose data, may be transmitted by the communication unit to the external device. The dose data may be used for a dose log or dose history established in the external device.

In the following, the wireless communications interface will be described referring to the example of Bluetooth® communication between the module and a smartphone. However, this is not to be understood as a limitation excluding the above-mentioned alternatives of wireless communication. In addition, the use of at least one LED (which may be either part of the sensor arrangement and/or which may be part of the electronic user feedback generator) is mentioned in the following as one possible example of an activity during which simultaneous activity of the communication unit is to be prevented. However, this is not to be understood as a limitation excluding the above-mentioned alternatives of activities of a sensor arrangement and/or an electronic user feedback generator.

The memory for storing measurement data, e.g. dose data, may be a separate memory or may be part of a main memory of the electronic module. These are controlled by the processor, which may for instance be the at least one microcontroller, a Digital Signal Processor (DSP), an Application Specific Integrated Circuit (ASIC), a Field Programmable Gate Array (FPGA) or similar. According to an aspect of the disclosure, the processor may be a von Neumann architecture processor, e.g., having a non-volatile main memory, for example a Flash main memory that contains both program code and data. In addition, there may be a volatile working memory, e.g., SRAM in small processors as suitable for the present disclosure, for data and intermediate calculations prior to transfer to a non-volatile long term data storage in main memory. Main memory may also be used to store a logbook on performed ejections/injections based on measurement data. Program memory may for instance be a Read-Only Memory (ROM), and main memory may for instance be a Random Access Memory (RAM).

The power source is connected to the processor and powers the processor and other components, like the sensor arrangement, the communication unit and the at least one electronic user feedback generator by way of a power supply. The power source may be a non-rechargeable, non-user replaceable coin cell.

It is common and desirable to want to use LEDs, the user feedback generator or other user output devices, at the same or similar time to Bluetooth® activity on the communication unit. This is because it is advantageous to give feedback to the user that Bluetooth® activity is about to start, is under way, or is complete. To mitigate the interaction of the output devices, for example the LED, the sequencing and detail control of Bluetooth® activity can be controlled to minimise unwanted battery voltage drops. This reduction in voltage drop can ultimately extend the life of the device by allowing the coin cell to run down to lower operating voltages without the voltage drops interrupting power and causing brown-outs or power-cycling.

The ways for achieving this according to the present disclosure include one or more of ensuring high load Bluetooth® activities do not occur during other activities such as the flashing of LEDs or transmission of audible signals from a sounder, limiting the amount of data sent to another device, e.g., a smartphone, at any one time by limiting the packet size and limiting the rate at which data is sent to another device, e.g., a smartphone. Some or all of these methods may be employed depending on the configuration or status of the device (e.g., available capacity of the coin cell). For instance, all three methods could be employed throughout the life of the device, or only once the coin cell capacity drops below a certain threshold. Thus, the useful life of the module may be extended beyond what would be achieved if such mitigations were not employed.

There are multiple ways of controlling the concurrency of activities causing large and/or prolonged current flows. According to an aspect of the present disclosure, the at least one processor is configured to inhibit data transfer by the communication interface for the exact time period that, e.g., an LED of the at least one electronic user feedback generator and/or, e.g., an LED of the sensor arrangement is activated. For example, where a large Bluetooth® data transfer occurs concurrently with an LED flash, the voltage drop is large. Inhibiting data transfer by the communication interface for the exact time period that the at least one electronic user feedback generator and/or the sensor arrangement is activated has the effect of mitigation to time the Bluetooth® data transfer after the LED flash. In this case the voltage drops do not add together and the maximum voltage drop of the Bluetooth® data transfer is controlled to be similar to that during a single LED flash.

In addition, or as an alternative, the at least one processor may be configured to inhibit data transfer by the communication interface for an extra time period after the at least one electronic user feedback generator and/or the sensor arrangement was activated. This allows the power source to recover.

Further, the at least one processor may be configured to conduct an operation routine comprising preset times for operation activities of the at least one electronic user feedback generator and/or the sensor arrangement, wherein the at least one processor is configured to the start data transfer by the communication interface only after said operation activities have ceased. In other words, a signal could be used a predetermined time after the last LED activity, or a predetermined time after a reference point where it is then known the LED activity will have ceased, in order to cease Bluetooth® activity inhibition or to trigger specific high load Bluetooth® activities.

For example, in Bluetooth® data transfer, the attribute protocol (ATT) defines the protocol of transferring the attribute data. A generic ATT packet comprises the OP-code (e.g., 1 byte) which indicates the ATT operation such as write command, notification, read response, etc., the attribute handle (e.g., 2 bytes) for identification of the data, and the ATT data field which contains the application data and the size of which depends on the ATT maximum transmission unit (MTU) of e.g., 23 bytes or 247 bytes or 512 bytes. Thus, as the overhead of OP-code and attribute handle of 3 bytes remains the same, use of a larger MTU size is typically preferred for fast transfer of high data volumes because the overhead in each transmission would be lower compared to splitting the data transfer in several smaller packets each having a (relatively) high overhead. Nevertheless, according to a further aspect of the present disclosure, the amount of data sent, e.g., to a smartphone, at the same time may be limited by limiting the packet size of the data transferred. In more detail, the at least one processor may be configured to not use the maximum available MTU size in order to reduce current drain during transmission. For example, the at least one processor may be configured to limit the amount of data transferred by the communication interface at any one time by setting the maximum transmission unit (MTU) to between 20 bytes and 100 bytes. For example, a lower limit for the MTU may be 23 bytes, 30 bytes or 40 bytes. As indicated above, a too small MTU size increases the overhead in each transmission which is undesired. On the other hand, an upper limit for the MTU size may be set to be below 80 bytes, or below 70 bytes, thereby reducing the voltage drop compared to larger MTU sizes. In an example, the MTU may be set to about or exactly 50 bytes. It has been observed that the voltage drop with an MTU of 247 bytes is significantly higher than the voltage drop caused by a transmission where the MTU is set (i.e., limited) to up to 50 bytes.

Another way of increasing data throughput is transfer of multiple (data) packets per connection event. However, it has been found that simultaneous transfer of a high number of packets may cause a significant voltage drop too. Thus, according to a further aspect of the present disclosure, the at least one processor may be configured to limit the rate at which data is sent, for example to limit the number of data packets sent simultaneously by the communication interface to another device to less than five packets at a time, for example to only one or two packets at a time. In addition or as an alternative, the at least one processor may be configured to set the time between two packet transmissions by the communication interface to another device to be below one data packet every 5 ms, for example to transfer one data packet every 10 ms to every 30 ms, for example every 20 ms. In other words, there are two control options described here to limit the rate of data transfer. The first is to control and limit the number of data packets sent simultaneously and the second is to control and the time between packet transmissions. For example, comparing the voltage behaviour arising from limiting the transfer to one packet at a time and a time gap of 20 ms between individual transfers) with the voltage behaviour from transferring five packets at a time (with the same 20 ms time interval), in the latter case the voltage drop is significantly higher. Further, a much reduced voltage drop may be observed when comparing the voltage behaviour associated with transferring one data packet every 5 ms with the voltage behaviour where one data packet is transferred every 20 ms.

According to a further aspect of the present disclosure, a method for operating an electronic module for a drug delivery device is provided. The drug delivery device may comprise a dose setting and drive mechanism that is configured to perform a dose setting operation for setting a dose to be delivered by the drug delivery device and a dose delivery operation for delivering the set dose. The electronic module may comprise at least one processor, a sensor arrangement connected to the at least one processor and operable to generate measurement data indicative of the dose setting operation and/or the dose delivery operation, a communication unit with a wireless communication interface connected to the at least one processor and operable to establish communication with another device and to transfer data to another device, at least one electronic user feedback generator connected to the at least one processor and operable to generate a feedback signal, a memory for storing measurement data, and power source connected to the at least one processor.

For example, the method comprises the following steps:
a) generating measurement data indicative of the dose setting operation and/or the dose delivery operation by the sensor arrangement,
b) storing said measurement data in the memory,
c) establishing communication with another device by means of the communication unit and transferring data to said device,
d) providing a feedback signal to a user by means of the at least one electronic user feedback generator.

In the method according to the present disclosure, the at least one processor may prevent data transfer by the communication interface during step a) and/or during step d), and/or limit the amount of data transferred by the communication interface at any one time by limiting the packet size, and/or limit the rate at which data is transferred by the communication interface.

According to a further aspect, the present disclosure is directed to a computer program adapted to execute the above-mentioned method when implemented in a processor of an electronic module, e.g., the above-mentioned electronic module, the computer program comprising computer program means for: preventing data transfer by the communication interface during generation of measurement data indicative of the dose setting operation and/or the dose delivery operation by the sensor arrangement and/or during generation of a feedback signal to a user by means of the at least one electronic user feedback generator, and/or limiting the amount of data transferred by the communication interface at any one time by limiting the packet size, and/or limiting the rate at which data is transferred by the communication interface.

The present disclosure further pertains to a drug delivery device comprising the electronic module as described above. The drug delivery device for delivery of a medicament may comprise a dose setting and drive mechanism which is configured to perform a dose setting operation for setting a dose to be delivered by the drug delivery device and a dose delivery operation for delivering the set dose and which comprises a first member. The drug delivery device may further comprise a container receptacle which is releasably attached to the dose setting and drive mechanism. As an alternative, the container receptacle may be permanently attached to the dose setting and drive mechanism. The container receptacle is adapted to receive a container, e.g., a cartridge, containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des (B30) human insulin, Lys(B29) (N-tetradecanoyl)-des(B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope of the present disclosure, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g., the axis which extends through the proximal and distal ends of the cartridge, the cartridge holder or the drug delivery device.

### BRIEF DESCRIPTION OF THE FIGURES

Non-limiting, exemplary embodiments of the disclosure will now be described with reference to the accompanying drawings, in which:

FIG. 1 shows an embodiment of a drug delivery device of the present disclosure;

FIG. 2 schematically illustrates an embodiment of an electronic module for a drug delivery device of the present disclosure;

FIG. 3 schematically illustrates an example of LED activity and Bluetooth activity over time;

FIG. 4 schematically illustrates a further example of LED activity and Bluetooth activity over time;

FIG. 5 schematically illustrates a further example of LED activity and Bluetooth activity over time; and

FIG. 6 schematically illustrates an example of LED activity and an LED end flag over time.

### DETAILED DESCRIPTION

In the figures, identical elements, identically acting elements or elements of the same kind may be provided with the same reference numerals.

In the following, some embodiments will be described with reference to an insulin injection device. The present disclosure is however not limited to such application and may equally well be deployed with injection devices that are configured to eject other medicaments or drug delivery devices in general, preferably pen-type devices and/or injection devices.

Embodiments are provided in relation to injection devices, in particular to variable dose injection devices, which record and/or track measurement data on doses delivered thereby. These data may include the size of the selected dose and/or the size of the actual delivered dose, the time and date of administration, the duration of the administration and the like. Features described herein include power management techniques (e.g., to facilitate small batteries and/or to enable efficient power usage).

Certain embodiments in this document are illustrated with respect to the injection device disclosed in EP 2 890 435 where an injection button and grip (dose setting member or dose setter) are combined. The injection button may provide the user interface member for initiating and/or performing a dose delivery operation of the drug delivery device. The grip or knob may provide the user interface member for initiating and/or performing a dose setting operation.

These devices are of the dial extension type, i.e., their length increases during dose setting. Other injection devices with the same kinematic behaviour of the dial extension and button during dose setting and dose expelling operational mode are known as, for example, the Kwikpen® device marketed by Eli Lilly and the Novopen® 4 device marketed by Novo Nordisk. An application of the general principles to these devices therefore appears straightforward and further explanations will be omitted. However, the general principles of the present disclosure are not limited to that kinematic behaviour. Certain other embodiments may be conceived for application to an injection device as described e.g. in WO2004078239 where there are separate injection button and grip components/dose setting members. Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

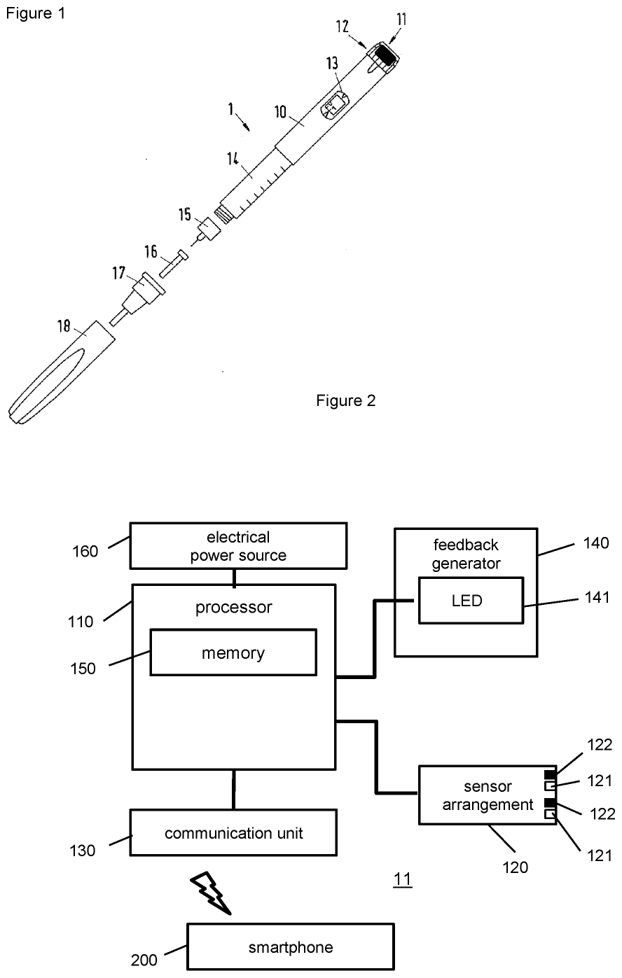

FIG. 1 is an exploded view of a medicament delivery device or drug delivery device. In this example, the medicament delivery device is an injection device 1, e.g., a pen-type injector, such an injection pen disclosed in EP 2 890 435.

The injection device 1 of FIG. 1 is an injection pen that comprises a housing 10 and contains a container 14, e.g., an insulin container, or a receptacle for such a container. The container may contain a drug. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialed in’ by turning a dosage knob 12, and a currently programmed or set dose is then displayed via dosage window 13, for instance in multiples of units. The indicia displayed in the window may be provided on a number sleeve or dial sleeve. For example, where the injection device 1 is configured to administer human insulin, the dosage may be displayed in so-called International Units (IU), wherein one IU is the biological equivalent of about 45.5 micrograms of pure crystalline insulin ( 1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve assembly that is configured to move when the dial grip 12 is turned, to provide a visual indication of a currently set dose. The dial grip 12 is rotated on a helical path with respect to the housing 10 when setting a dose.

In this example, the dial grip 12 includes one or more formations to facilitate attachment of a data collection device. Especially, the dial grip 12 may be arranged to attach or integrate an electronic (button) module 11 onto the dial grip 12. As an alternative, the dial grip may comprise such a button module of an electronic system.

The injection device 1 may be configured so that turning the dial grip 12 causes a mechanical click sound to provide acoustic feedback to a user. In this embodiment, the dial grip 12 also acts as an injection button. When needle 15 is stuck into a skin portion of a patient, and then dial grip 12 and/or the attached module 11 is pushed in an axial direction, the insulin dose displayed in display window 13 will be ejected from injection device 1. When the needle 15 of injection device 1 remains for a certain time in the skin portion after the dial grip 12 is pushed, the dose is injected into the patient's body. Ejection of the insulin dose may also cause a mechanical click sound, which may be different from the sounds produced when rotating the dial grip 12 during dialing of the dose.

In this embodiment, during delivery of the insulin dose, the dial grip 12 is returned to its initial position in an axial movement, without rotation, while the dial sleeve assembly is rotated to return to its initial position, e.g., to display a dose of zero units. FIG. 1 shows the injection device 1 in this 0 U dialed condition. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g., 28 days after the first use) is reached. In the case of a reusable device, it is possible to replace the insulin container.

Furthermore, before using injection device 1 for the first time, it may be necessary to perform a so-called “prime shot” to remove air from insulin container 14 and needle 15, for instance by selecting two units of insulin and pressing dial grip 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user. Nevertheless, differences (e.g., losses) between the ejected amounts and the injected doses may need to be taken into account.

As explained above, the dial grip 12 also functions as an injection button so that the same component is used for dialling/setting the dose and dispensing/delivering the dose. As an alternative (not shown), a separate injection button may be used which is axially displaceable, at least a limited distance, relative to the dial grip 12 to effect or trigger dose dispensing.

In the following, an electronic module 11 according to the present disclosure will be described with respect to exemplary embodiments and with reference to FIGS. 1 to 6. In FIG. 1, the electronic module 11 is depicted as being integrated in the proximal end of the injection device 1, specifically integrated into the dial grip/dose button 12. As an alternative, the electronic module 11 may be a separate component part which may be permanently or releasably attached to the injection device 1, e.g., to the grip/dose button 12.

As depicted in FIG. 2, an exemplary electronic module comprises a processor 110, a sensor arrangement 120, a communication unit 130, an electronic user feedback generator 140, a memory 150, and a power source 160.

In the example depicted in FIG. 2, the sensor arrangement 120 is connected to the processor 110 and operable to generate measurement data indicative of the dose setting operation and/or the dose delivery operation. For this purpose the sensor arrangement comprises a LED 121 and a photo detector 122, together forming an optical sensor. Alternative sensor types could be implemented in addition to LED 121 and photo detector 122 or as an alternative thereto. Such alternative sensor types may include but are not limited to optical sensors, acoustic sensors, capacitive sensors, electrical switches.

The communication unit 130 comprises with a wireless Bluetooth® communication interface connected to the processor 110 and operable to establish communication with another (external) device, e.g., a smartphone 200. Further, the communication unit 130 is operable to transfer data, e.g., measurement data, to said other device 200.

The electronic user feedback generator 140 connected to the processor 110 and operable to generate a feedback signal to a user. In the exemplary arrangement of FIG. 2, the electronic user feedback generator 140 comprises an LED 141 for generating optical feedback signals. In addition to the LED 141 or as an alternative to the LED 141, the electronic user feedback generator 140 may comprise a sounder and/or a vibration generator.

The memory 150 is adapted for storing measurement data and is connected to the processor 110 or is integrated into the processor 110.

The power source 160 is connected to the processor 110. For example, the power source 160 is a non-rechargeable, non-user replaceable coin cell.

In addition to the Bluetooth® communication unit 130, the module 100 comprises LEDs 121 and 141 and, optionally may further comprise sounders, microphones, vibration generators or sensors for various purposes. These components may place a significant demand on the coin cell 160, which in combination with the Bluetooth® activity, could cause undesired voltage drops, especially if the activity of the other components coincides with Bluetooth® activity. It is common and desirable to want to use LEDs 121, 141, or other user output devices, at the same or similar time to Bluetooth® activity on the radio system. This is because it is advantageous to give feedback to the user that Bluetooth® activity is about to start, is under way, or is complete.

To mitigate the interaction of the output devices, for example LEDs 121, 141, the sequencing and detail control of Bluetooth® activity can be controlled to minimise the unwanted voltage drops. This reduction in voltage drop can ultimately extend the life of the module 11 by allowing the coin cell 160 to run down to lower operating voltages without the voltage drops interrupting power and causing brown-outs or power-cycling. Different ways for achieving this are described below. These ways may include ensuring high load Bluetooth® activities do not occur during other activities such as the flashing of LEDs 121, 141 or transmission of audible signals from a sounder, limiting the amount of data sent to the smartphone 200 at any one time by limiting the packet size and/or limiting the rate at which data is sent to the smartphone 200. Some or all of these methods may be employed depending on the configuration or status of the module 11, e.g., available capacity of the coin cell 160. For instance, all three methods could be employed throughout the life of the module 11, or only once the coin cell 160 capacity drops below a certain threshold. Thus the useful life of the module 11 may be extended beyond what would be achieved if such mitigations were not employed.

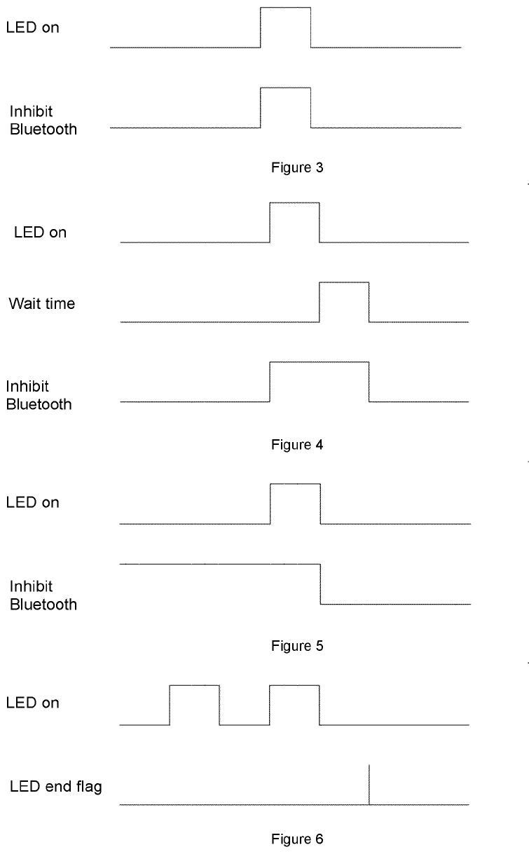

In FIG. 3 it is shown that the Bluetooth® activity could be inhibited for the exact time period that the LED, for example LED 121 and/or LED 141, is active (for example a flash). In FIG. 4 it is shown that the Bluetooth® activity could be inhibited for an extra time period after each LED activity, for example to allow the coin cell to recover. In FIG. 5 the Bluetooth® activity could be inhibited until after a specified LED activity. In FIG. 6a signal could be used a predetermined time after the last LED activity, or a predetermined time after a reference point (where it is then known the LED activity will have ceased), in order to cease Bluetooth® activity inhibition or to trigger specific high load Bluetooth® activities.

Further, the processor 110 is configured, i.e. suitable and adapted, to limit the amount of data sent to the smartphone 200 at any one time by limiting the packet size. For example, the voltage drop during a data transfer where the packets of data have been specifically controlled, e.g., the maximum transmission unit (MTU) is set to 50 bytes results in a moderate voltage drop, whereas the voltage drop is significantly higher where the packet size is not limited and has been set to the maximum value in this setup, an MTU of 247.

Still further, the processor 110 is configured to limit the rate at which data is sent to the smartphone 200. There are two control options described here to limit the rate of data transfer. The first is to control and limit the number of data packets sent simultaneously and the second is to control the time between packet transmissions. For example, the voltage behaviour arising from limiting the transfer to one packet at a time and a time gap of 20 ms between individual transfers results in a smaller voltage drop compared to the voltage behaviour from transferring five packets at a time with the same 20 ms time interval. In addition, the voltage behaviour associated with transferring one data packet every 5 ms shows a significantly increased voltage drop compared to an example where one data packet is transferred every 20 ms.

The response to request from the smartphone 200 to transfer records from memory 150 is delayed until after it has been determined that the LEDs 121, 141 have stopped flashing. This response has the potential to be the largest data transmission from the injection device 1 module 11 as it could contain the entire contents of the dose history. Connection requests and all other data communication is permitted up until this point in order to minimise the risk of missing connection events or triggering timeout events on the smartphone 200. The MTU is limited to 119 bytes. The time is controlled to be 20 ms between the transmission of individual data packets.

Although described mainly with respect to a drug delivery device having a similar working principle as the device disclosed in EP 2 880 435, the electronic module is applicable to any other type of drug delivery device having component parts performing a relative axial and/or rotational movement in defined conditions or states.

1-15. (canceled)

16. An electronic module for a drug delivery device comprising a dose setting and drive mechanism configured to perform a dose setting operation for setting a dose to be delivered by the drug delivery device and a dose delivery operation for delivering a set dose, the electronic module comprising:
at least one processor;
a sensor arrangement connected to the at least one processor and operable to generate measurement data indicative of the dose setting operation and/or the dose delivery operation;
a communication unit with a wireless communication interface connected to the at least one processor and operable to establish communication with another device and to transfer data to the other device;
at least one electronic user feedback generator connected to the at least one processor and operable to generate a feedback signal;
a memory for storing measurement data; and
a power source connected to the at least one processor;
wherein the at least one processor is configured to prevent data transfer by the communication interface during operation of the at least one electronic user feedback generator and/or during operation of the sensor arrangement, limit an amount of data transferred by the communication interface at any one time by limiting a packet size of the data transferred by the communication interface, and/or limit a rate at which data is transferred by the communication interface.

17. The electronic module of claim 16, wherein the at least one electronic user feedback generator comprises at least one light source.

18. The electronic module of claim 16, wherein the at least one electronic user feedback generator comprises at least one sounder.

19. The electronic module of claim 16, wherein the at least one electronic user feedback generator comprises at least one vibration generator.

20. The electronic module of claim 16, wherein the wireless communication interface is a Bluetooth® interface.

21. The electronic module of claim 16, wherein the sensor arrangement comprises at least one light source and at least one light sensor.

22. The electronic module of claim 16, wherein the at least one processor is configured to inhibit data transfer by the communication interface for an exact time period that the at least one electronic user feedback generator and/or the sensor arrangement is activated.

23. The electronic module of claim 16, wherein the at least one processor is configured to inhibit data transfer by the communication interface for an extra time period after the at least one electronic user feedback generator and/or the sensor arrangement was activated.

24. The electronic module of claim 16, wherein the at least one processor is configured to conduct an operation routine comprising preset times for operation activities of the at least one electronic user feedback generator and/or the sensor arrangement.

25. The electronic module of claim 24, wherein the at least one processor is configured to start the data transfer by the communication interface only after the operation activities have ceased.

26. The electronic module of claim 16, wherein the at least one processor is configured to not use the maximum available MTU size to reduce current drain during transmission.

27. The electronic module of claim 16, wherein the at least one processor is configured to limit a number of data packets sent simultaneously by the communication interface to the other device to less than five packets at a time.

28. The electronic module of claim 16, wherein the at least one processor is configured to set the time between two packet transmissions by the communication interface to the other device to be below one data packet every 5 ms.

29. The electronic module of claim 16, wherein the power source is a non-rechargeable, non-user replaceable, coin cell.

30. A drug delivery device for delivery of a medicament, the drug delivery device comprising:
an electronic module comprising:
at least one processor;
a sensor arrangement connected to the at least one processor and operable to generate measurement data indicative of a dose setting operation and/or a dose delivery operation;
a communication unit with a wireless communication interface connected to the at least one processor and operable to establish communication with another device and to transfer data to the other device;
at least one electronic user feedback generator connected to the at least one processor and operable to generate a feedback signal;
a memory for storing measurement data; and
a power source connected to the at least one processor;
a dose setting; and
a drive mechanism configured to perform a dose setting operation for setting a dose to be delivered by the drug delivery device and a dose delivery operation for delivering a set dose, the drive mechanism comprising:
a first member; and
a container receptacle connected to the dose setting and drive mechanism and adapted to receive a container containing a medicament.

31. The drug delivery device of claim 30, wherein the container receptacle is permanently connected to the dose setting and drive mechanism.

32. The drug delivery device of claim 30, wherein the container receptacle is releasably connected to the dose setting and drive mechanism.

33. The drug delivery device of claim 30, wherein the at least one processor is configured to prevent data transfer by the communication interface during operation of the at least one electronic user feedback generator and/or during operation of the sensor arrangement.

34. The drug delivery device of claim 30, wherein the at least one processor is configured to limit an amount of data transferred by the communication interface at any one time by limiting a packet size of the data transferred by the communication interface, and/or limit a rate at which data is transferred by the communication interface.

35. A method for operating an electronic module for a drug delivery device, the drug delivery device comprising a dose setting and drive mechanism configured to perform a dose setting operation for setting a dose to be delivered by the drug delivery device and a dose delivery operation for delivering a set dose, wherein the electronic module comprises:
at least one processor;
a sensor arrangement connected to the at least one processor and operable to generate measurement data indicative of the dose setting operation and/or the dose delivery operation;
a communication unit with a wireless communication interface connected to the at least one processor and operable to establish communication with another device and to transfer data to the other device;
at least one electronic user feedback generator connected to the at least one processor and operable to generate a feedback signal;
a memory for storing measurement data; and
a power source connected to the at least one processor;
the method comprising:
generating measurement data indicative of the dose setting operation and/or the dose delivery operation by the sensor arrangement;
storing the measurement data in the memory;
establishing communication with the other device by means of the communication unit and transferring data to the other device;
providing a feedback signal to a user by means of the at least one electronic user feedback generator;
wherein the at least one processor prevents data transfer by the communication interface during generating measurement data and/or during providing the feedback signal, and/or limits an amount of data transferred by the communication interface at any one time by limiting a packet size, and/or limits a rate at which data is transferred by the communication interface.
